(19) United States
(12) Patent Application Publication
(10) Pub. No.: US 2010/0173027 A1
(43) Pub. Date: Jul. 8, 2010

(54) O/W EMULSION FOR HAND CARE

(75) Inventors: Rainer Kroepke, Schenefeld (DE); Ingo Hahn, Pinneberg (DE); Christel Lemm, Buxtehude (DE)

Correspondence Address: GREENBLUM & BERNSTEIN, P.L.C., 1950 ROLAND CLARKE PLACE, RESTON, VA, 20191, US

(73) Assignee: Beiersdorf AG, Hamburg (DE)

(21) Appl. No.: 12/303,560

(22) PCT Filed: May 24, 2007
(86) PCT No.: PCT/EP07/55032

§ 371 Date: Feb. 11, 2009

(30) Foreign Application Priority Data
Jun. 8, 2006 (DE) 10 2006 027 138.6

(51) Int. Cl. A61K 31/047; A61K 36/28; A61K 8/97; A61P 17/02; A61Q 19/00; A61K 8/34

(52) U.S. Cl. 424/764; 514/738

(57) ABSTRACT

An O/W emulsion which comprises one or more fatty alcohols, one or more glycerol esters of fatty acids and at least 15% by weight, based on the total weight of the emulsion, of at least one moisturizer. Also disclosed is a cosmetic composition which comprises the emulsion.

The invention describes an O/W emulsion comprising fatty alcohol and/or fatty alcohol mixtures, at least one moisturizer and at least one glycerol ester of a fatty acid. The proportion of moisturizer, preferably glycerol, is at least 15% by weight, based on the total weight of the emulsion.

The emulsion is particularly suitable for the preparation of an agent for the immediate care, beautification and/or treatment of rough, cracked and/or inflamed skin, preferably of the hand. Preferred embodiments of the emulsion according to the invention show an “instant effect” on very dry skin or rough/cracked skin.

Numerous hand creams, lotions and gels are known that can be applied to human skin and preferably to the hand. For example, Lindesa O hand cream, Logona-Daily Care hand cream or pH5 Eucerin hand cream. The hand creams are suitable for care after work in the case of dry and degreased skin, for the intensive care of rough and worn hands. The package advertising of hand creams available on the market are manifold, such as, e.g., “Gives your hands protection, care and moisture even under difficult conditions such as cold or other adverse environmental effects.”

The known emulsion-based hand creams often contain, in addition to water, glycerol, cetearyl alcohol and glyceryl stearate.

It would therefore be desirable to provide an emulsion to be applied to the skin, preferably of the hand, which causes an immediate care and/or beautifying effect in the case of rough, cracked or dry skin.

It is furthermore desirable to provide, in addition to the known care products, a further emulsion-based cosmetic preparation that enriches the selection of the preparations available on the market.

The invention comprises an O/W emulsion comprising fatty alcohol and/or fatty alcohol mixtures, at least one glycerol ester of a fatty acid and at least one moisturizing agent.

According to the invention, the moisturizing agent, ideally glycerol, is present in a high concentration, preferably more than 20% by weight. This leads to a very good and lasting moisturization in and on the skin. In this high concentration, the moisturizing agent, preferably glycerol, also has softening properties on the skin to which it is applied.

Other treatment agents that can preferably be added to the emulsion can thereby penetrate better and more easily into the cracked or rough skin and thus provide a sustained support of the treatment effect.

It has hitherto been a disadvantage that a high proportion of moisturizing agents in emulsions resulted in care emulsions having an unacceptable consistency and a undesirable stickiness for the user.

This disadvantage was surprisingly overcome according to the invention. Through the combination of at least one glycerol ester, in particular glyceryl laurate, fatty alcohol or fatty alcohol mixtures and a high proportion of moisturizing agents of more than 15% by weight, in particular more than 20% by weight, an improvement in the consistency and above all in the stickiness of the emulsion was achieved.

The use is preferably given thereby of a combination of at least one fatty acid glycerol ester, in particular glyceryl laurate, at least one fatty alcohol and more than 15% by weight of moisturizing agents, in particular glycerol, for improving the consistency and/or the stickiness of cosmetic preparations.

Skin moisture is a term from the cosmetics industry. Healthy skin has a natural moisture content. Only when the human skin exhibits anomalies with respect to dryness, does a lack of skin moisture have an effect. Apart from pathological causes, the age of human skin as well as the pigmentation play a role. Humectant factors such as, e.g., urea, play an important role in the moisture of the skin. These can be supplied to the skin by skin care compositions.

Normally, human skin does not need any auxiliary agents to maintain natural moisture. However, an unhealthy way of life, dry air (particularly in solaria and heated interior rooms), environmental impact, stress and lengthy sun-bathing contribute to the removal of moisture. Long, hot baths and detergent residue in clothing also cause the loss of important constituents of the hydrolipid system of the skin. In order to prevent the drying out of skin, moisturizing creams are used. Also regreasing soaps and largely soap-free detergents return the fat to the skin. It thus becomes smoother and supple again.

Here in particular body cleansing agents are recommended that do not destroy the natural hydrolipidic film of the skin (pH of the skin=5.5).

In some dwellings where ventilation is not possible as usual because of negative environmental conditions, so-called air humidifiers can also be used to support a good skin moisture.

Through the provision of the emulsion according to the invention, the user now has another option available for treating dry, rough or cracked skin, in particular of the hand, and of supplying moisture to it.

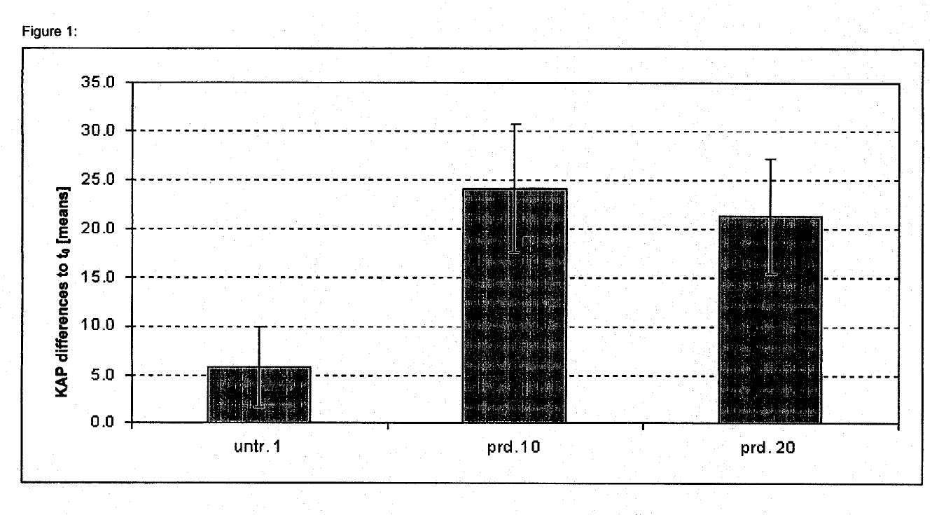

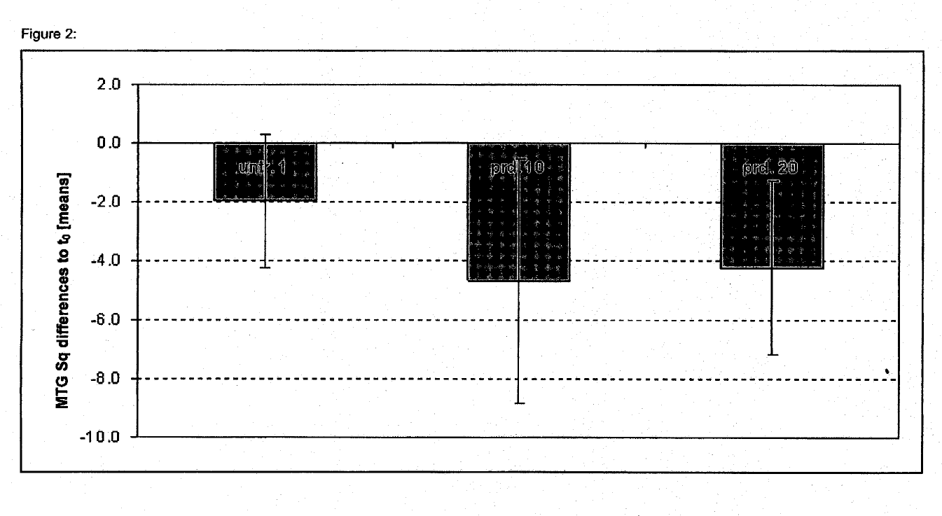

Potency tests, as shown in FIGS. 1 and 2, support the skin-moisturizing capacity (KAP) of the emulsion according to the invention (prod 10) compared to untreated skin (untr. 1) and compared to products from the prior art (prod 20, Neutrogena® hand cream concentrate). The emulsion according to the invention, with up to 30% by weight of glycerol, increases the skin moisture by 417% (FIG. 1).

Values were likewise determined for skin-smoothing, which provide evidence that the emulsion according to the invention can be used effectively for cosmetic care and beautification of the skin, in particular for dry, rough or cracked skin. The values show that the emulsion according to the invention improves the smoothness of the skin by 235% after 7 days (FIG. 2).

The studies carried out show an increase in effectiveness in the skin care due to the improved moisture of the skin as well as a reduced roughness of the skin. The emulsions according to the invention can therefore be used to improve or enhance the moisture of the skin (KAP) and/or smoothness of the skin (MTG).

An improvement of the moisture of the skin (KAP) by up to 417% and/or an increase in the smoothness of the skin (MTG) by up to 235% is possible with the aid of the emulsion that is preferred according to the invention.

The studies on a. Skin Moisture (parameter code: KAP) and b. Skin Roughness (parameter code: MTG) were carried out as follows:

a. Skin Moisture (parameter code: KAP)

Measuring device: Corneometer CM 825 (Courage+Khazaka, Cologne, Germany)
Parameter: permittivity [a.u.]=arbitrary units, median resulting from repeated Measurement
Repetitions: 10 measurements per area
Guideline, stipulation: “EEMCO Guidance for the Assessment of Stratum Corneum Hydration: Electrical Methods” by E. Berardesca (published in Skin Research and Technology, 1997, 3: 126-132)

b. Skin Roughness (parameter code: MTG)

Measuring device: μCAD supplied by GFMesstechnik (Teltow, Germany)
Principle: Optical Topometry on silicone imprints (negative impressions of the topographic features of the skin made with Silflo®)
Parameter: root mean square deviation of the surface sq [μm], original values
Repetition: 1 imprint per area
Guideline, stipulation: “EEMCO* guidance for the assessment of skin topography” from J. L. Lév\tilde{e}que (published in J Eur Acad Dermatol Venerol, 1999, 12: 103-114

The emulsions can be used to improve or enhance the skin moisture (KAP) and/or skin smoothness (MTG).

The emulsion according to the invention is thus preferably to be used to prepare a composition for the care or alleviation of rough, cracked and/or inflamed skin, preferably of the hand.

In a preferred embodiment the emulsion comprises at least 20% by weight, preferably at least 25% by weight of moisturizer, based on the total weight of the emulsion.

Moisturizer is the term used to refer to substances or mixtures of substances which impart to cosmetic or dermatological preparations the property, following application or distribution on the surface of the skin, of reducing moisture release by the horny layer (also called trans-epidermal water loss (TEWL)) and/or of positively influencing hydration of the horny layer.

Advantageous moisturizers for the purposes of the present invention are, for example, glycerol, lanolin and esters thereof, lanolin alcohol, sorbitol, lactic acid and salts thereof, pyrrolidone carboxylic acid and salts thereof, and urea. Further moisturizers are, for example, polymeric moisturizers from the group of polysaccharides which are water-soluble and/or water-swellable and/or are gellable by means of water. Hyaluronic acid, chitosan and/or a fucose-rich polysaccharide, which is filed in Chemical Abstracts under the registry number 178463-23-5 and which is available, for example, under the name Fucogel® 1000 by SOLABIA S. A., for example, are particularly advantageous.

Glycerol is the preferred moisturizer. The proportion of glycerol in the emulsion advantageously is at least 20% by weight, preferably at least 25% by weight, based on the total weight of the emulsion.

Glycerol has proven to be a very effective moisturizer, because inter alia with these high proportions, it results in a measurable immediate effect with respect to skin moisturizing and skin smoothing.

The proportion of fatty alcohol or fatty alcohol mixtures is preferably at least about 2% by weight, based on the total weight of the emulsion.

Preferably, the proportion of fatty alcohol or fatty alcohol mixtures is no more than about 10% by weight, based on the total weight of the emulsion. It has proven to be particularly advantageous if the fatty alcohols or fatty alcohol mixtures have a melting point in the range of about 32° C. to 56° C.

Through the combination with fatty alcohols or fatty alcohol mixtures, in particular with a melting point of about 32-56° C., a very good physical emulsion stability and a “melting” behavior of the texture on the skin are achieved, which in turn substantially moderates the otherwise immense stickiness of an emulsion with these high glycerol concentrations.

In another aspect of the invention, the proportion of fatty acid glycerol ester is preferably at least about 0.1% by weight, preferably at least about 0.5% by weight, based on the total weight of the emulsion.

Preferably the proportion of fatty acid glycerol ester is preferably no more than about 5% by weight, preferably no more than about 3% by weight, based on the total weight of the emulsion.

The fatty acid glycerol esters with a C chain length of less than 22 carbon atoms are to be selected as particularly advantageous. The preferred fatty acid glycerol ester is glyceryl laurate.

The combination of fatty alcohol or fatty alcohol mixtures, in particular with a melting point of about 32-56° C., fatty acid glycerol ester, in particular glyceryl laurate, and more than 15% by weight of moisturizers, in particular glycerol, in an O/W emulsion leads to an improvement in the skin moisture and skin smoothness (see test, FIGS. 1 and 2) and at the same time to a reduction of the stickiness and thus to a more pleasant application and handling.

In another preferred aspect, the O/W emulsion according to the invention comprises
at least about 2% by weight, preferably no more than about 10% by weight of fatty alcohol and/or fatty alcohol mixtures having a melting point in the range from about 32° C. to 56° C.,
at least about 0.1% by weight, preferably at least about 0.5% by weight, preferably no more than 5% by weight of fatty acid glycerol ester having a C chain length of less than 22 carbon atoms, preferably glyceryl laurate, and
at least 15% by weight, in particular at least 20% by weight, preferably at least 25% by weight of a moisturizer, preferably glycerol.

The proportions are each based on the total weight of the emulsion.

In a preferred aspect of the invention, at least one phosphate emulsifier can be added to the emulsion. The content of phosphate emulsifiers is preferably selected in the range of from at least about 0.1, preferably at least 0.5% by weight, based on the total weight of the emulsion.

The content of phosphate emulsifiers is preferably no more than about 5% by weight, preferably no more than about 2.5% by weight, based on the total weight of the emulsion.

Trilaureth-4 phosphate and/or triceteareth-4 phosphate are preferably selected as phosphate emulsifiers.

The combination of the constituents according to the invention with at least one phosphate emulsifier exhibits a stabilizing effect on O/W emulsions with very high moisturizers. Furthermore, the phosphate emulsifiers provide a very soft, silky feeling on the skin and improve the distributability of the texture.

This process is comparable with lecithin, chemical name: phosphatidyl choline, a phosphoglyceride, which is used in the food industry as an emulsifier for stabilizing fat-in-water mixtures.

In another preferred aspect, the O/W emulsion according to the invention comprises
at least about 2% by weight, preferably no more than 10% by weight of fatty alcohol and/or fatty alcohol mixtures having a melting point in the range of from about 32° C. to 56° C.,
at least about 0.1% by weight, preferably at least about 0.5% by weight, preferably no more than about 5% by weight, of fatty acid glycerol ester with a C chain length of less than 22 carbon atoms, preferably glyceryl laurate
at least about 0.1% by weight, preferably at least about 0.5% by weight, preferably no more than about 5% by weight of phosphate emulsifiers, preferably trilaureth-4 phosphate and/or triceteareth-4 phosphate, and
at least 15% by weight, in particular at least 20% by weight, preferably at least 25% by weight of a moisturizer, preferably glycerol.

The proportion are each based on the total weight of the emulsion.

In a preferred aspect, further skin care, wound-healing, anti-inflammatory and/or pain-relieving substances can be added to the O/W emulsion according to the invention.

Panthenol and/or calendula oil are preferably selected.

An emulsion to which panthenol in a proportion of about 0.1 to 5% by weight, preferably about 0.5 to 2% by weight, and calendula oil in the range of about 0.01 to 5% by weight, preferably 0.1 to 4% by weight, based on the total weight of the emulsion, are added, is particularly preferred.

In another preferred aspect, the O/W emulsion according to the invention comprises therefore
2 to 10% by weight of fatty alcohol and/or fatty alcohol mixtures having a melting point in the range of from about 32° C. to 56° C.,
0.1 to 5% by weight, preferably 0.5% to 3% by weight, of fatty acid glycerol ester with a C chain length of less than 22 carbon atoms, preferably glyceryl laurate
at least 15% by weight, in particular at least 20% by weight, preferably at least 25% by weight of a moisturizer, preferably glycerol.
optionally phosphate emulsifiers, preferably trilaureth-4 phosphate and/or triceteareth-4 phosphate
0.1 to 5% by weight, preferably 0.5 to 2% by weight, of panthenol and
about 0.01 to 5% by weight, preferably 0.1 to 4% by weight calendula oil.

Panthenol, also known under the names dexpanthenol, pantothenol, D-pantothenyl alcohol or provitamin B5, is described by the chemical names (R)-2,4-dihydroxy-N-(3-hydroxypropyl)-3,3-dimethylbutanamide, (R)-2,4-dihydroxy-3,3-dimethylbutyric acid-3-hydroxypropyl amide.

Panthenol is the alcoholic and, in cosmetics, the more stable form of the B vitamin pantothenic acid. Panthenol is converted into pantothenic acid in the skin. In skin care preparations it is to provide an improved skin moisture, in that it penetrates deep into the lower layers of the skin and binds water there. Panthenol develops this effect in addition to its normal function in the organism.

Panthenol (dexpanthenol) promotes the energy metabolism of the skin cells and the optimal nourishment thereof. Similar to vitamin A, it stimulates the division of the skin cells. Since it calms the skin and has a healing effect, it also helps in the case of acute sunburn. In addition, panthenol has a very special importance for the hair.

Calendula oil, botanical name calendula officinalis L, comes from the Asteraceae family of plants—composites. The marigold is behind the name of the calendula. Ointments, tea and other remedies have been produced from it for centuries. The Latin name “calendula” comes from the Latin word “calendae,” the first day of the month, and since the marigold blooms in many “calendis” i.e. months, it was called calendula.

Calendula oil is a maceration that is usually extracted in olive oil or soy bean oil. Macerations are vegetable oils in which a medicinal plant in an oil is exposed to the sun for a period and its active ingredients have been transferred to the vegetable oil. Soy bean oil as a constituent of treatment emulsions is therefore often merely the “carrier” for calendula oil.

Marigold oil, calendula oil, has wound-healing, anti-inflammatory and pain-relieving effects. It is particularly helpful with dry and cracked skin, but also treats delicate and sensitive skin. It supports the healing process in the case of rheumatic complaints and wounds that heal poorly.

Based on the advantages and ingredients described, the emulsions according to the invention advantageously represent the basis of a cosmetic preparation. According to the invention, the term emulsion also covers cosmetic preparations based on a corresponding emulsion.

The emulsion or the preparation containing it can also advantageously be present in a form suitable for topical application. For example, the preparation can be present in the form of a cream, a lotion, a gel, an ointment, a tincture, a milk, a balm, a plaster impregnated with the preparation, a wipe impregnated with the preparation, a textile impregnated with the preparation, a pad impregnated with the preparation, a spray, an aerosol, a roll-on, a stick, a soft solid, a powder or a powder spray.

The emulsion is preferably to be used for application to human skin, preferably of the hand. It is therefore preferably offered and used as a hand cream.

The emulsion according to the invention can advantageously be used for the care and/or beautification of dry, rough, cracked and/or inflamed skin, preferably of the hand.

It was surprisingly found that the emulsion according to the invention immediately calms the skin to be treated and quickly reestablishes the disturbed moisture balance of the skin in a lasting manner.

According to the invention, “instant effect” describes an improvement in the skin moisture or skin smoothness within one hour up to 7 days. This in particular after a single or regular application of the emulsion according to the invention in sufficient quantity on the skin to be treated.

A sufficient quantity is the quantity usually applied to the corresponding areas of the skin. In the case of a hand cream this can be about amounts of 0.5 to 1 g, in the case of body lotions easily 10 times or 100 times that amount, wherein here the size of the area of skin to be treated also plays a role.

A method for improving skin moisture or skin smoothness is thus preferred. To this end, the emulsion according to the invention or a cosmetic preparation containing it is applied to the skin in sufficient quantity. After a single use as well as preferably after application once a day a surprising “instant effect” is shown within one hour as well as a lasting effect after one week.

According to the invention, improvement means an increase in or positive change in the skin moisture or skin smoothness with respect to the condition of the skin without the application of the emulsion according to the invention.

In particular, this “instant effect” appears with the cosmetic preparations comprising the O/W emulsion according to the invention with
2 to 10% by weight of fatty alcohol and/or fatty alcohol mixtures with a melting point in the range of from about 32° C. to 56° C.,
0.1% to 5% by weight, preferably 0.5% to 3% by weight, of fatty acid glycerol ester with a C chain length of less than 22 carbon atoms, preferably glyceryl laurate
at least 15% by weight, in particular at least 20% by weight, preferably at least 25% by weight of a moisturizer, preferably glycerol.
optionally, phosphate emulsifiers, preferably trilaureth-4 phosphate and/or triceteareth-4 phosphate
0.1 to 5% by weight, preferably 0.5 to 2% by weight, of panthenol and about 0.01 to 5% by weight, preferably 0.1 to 4% by weight, of calendula oil.

The emulsion is to be used as a cosmetic and can in addition be used to prepare a composition for the preferably immediate care, beautification and/or treatment of rough, cracked and/or inflamed skin, preferably of the hand. Dry and sensitive skin thus remains healthy, beautiful and soft longer.

The compositions according to the invention are not therapeutically active compositions.

The aqueous phase of the emulsion according to the invention or of preparations comprising it, can advantageously contain conventional cosmetic auxiliary agents, such as, for example alcohols, in particular those with a low C number, preferably ethanol and/or isopropanol, diols or polyols with a low C number and ethers thereof, preferably propylene glycol, ethylene glycol, ethylene glycol monoethyl ether or ethylene glycol monobutyl ether, propylene glycol monomethyl ether, propylene glycol monoethyl ether or propylene glycol monobutyl ether, diethylene glycol monomethyl ether or diethylene glycol monoethyl ether and analogous products, polymers, foam stabilizers and electrolytes, wherein these are not considered as the above-referenced moisturizers.

The emulsion according to the invention or cosmetic preparations used according to the invention can naturally contain cosmetic auxiliaries as are conventionally used in such preparations, for example, preservatives, bactericides, UV filters, antioxidants, water-soluble vitamins, minerals, suspended solid particles, perfumes, foam inhibitors, dyes, coloring pigments, thickeners, moisturizers and/or humectants, or other usual components of a cosmetic or dermatological formulation, such as alcohols, polyols, polymers, foam stabilizers, or silicone derivatives.

The following examples show various emulsions according to the invention or preparations containing them and the use thereof. The proportions given are percentages by weight based on the total weight of the preparation, unless stated otherwise:

### EXAMPLES

### O/W Emulsion

| | 1 | 2 | 3 | 4 | 5 |
|---|---|---|---|---|---|
| Glyceryl laurate | 1.0 | 2.0 | 1.0 | 0.5 | 0.75 |
| Ceteareth-3 | 1.0 | 0.75 | 2.0 | 2.5 | 0.5 |
| Triceteareth-4 phosphate | 0.1 | 0.75 | 1.2 | 1.7 | 2.5 |
| Petroleum (Vaseline) | 1.0 | 1.5 | 4.0 | 5.0 | 3.2 |
| Calendula oil | 0.1 | 0.5 | 0.1 | 3.5 | 1.75 |
| Dimethicone | 5.0 | 3.0 | 3.5 | 2.0 | 1.0 |
| Myristyl alcohol | 1.0 | 2.0 | 4.0 | 1.0 | 0.75 |
| Sorbitan stearate | 0.5 | 0.75 | 2.5 | 3.5 | 1.25 |
| Cetyl alcohol | 4.5 | 3.5 | 4.0 | 1.0 | 2.75 |
| Panthenol | 0.5 | 0.75 | 0.5 | 2.0 | 0.25 |
| Isopropylpalmitate | 0.5 | 1.0 | 2.5 | 3.0 | 7.5 |
| Perfume | q.s. | q.s | q.s | q.s | q.s |
| Methylparaben | 0.4 | 0.1 | 0.30 | 0.3 | 0.4 |
| Propylparaben | 0.1 | 0.05 | 0.15 | 0.15 | 0.1 |
| Phenoxyethanol | 0.3 | 1.0 | 0.7 | 0.5 | 0.1 |
| Glycerol | 15 | 16.5 | 26.5 | 36.5 | 46.5 |
| Sodium carbomer | 0.25 | 0.45 | 0.3 | 0.15 | 0.65 |
| Water | ad 100 | ad 100 | ad 100 | ad 100 | ad 100 |

Preferably useable as hand cream, foot cream, mousse application with/without propellant or lotion

### O/W Emulsion

| | 6 | 7 | 8 | 9 | 10 |
|---|---|---|---|---|---|
| Polyethylene | 1 | — | 2.5 | 2 | 1.5 |
| glycol(21)stearyl ether | | | | | |
| Polyethylene | 1 | — | 5.5 | 3 | 7.5 |
| glycol(2)stearyl ether | | | | | |
| Cetearyl glucoside | — | 8 | — | — | — |
| Glyceryl stearate | 0.1 | 0.5 | 3.0 | 2.5 | 5.0 |
| Dimethicone | 5.0 | 3.0 | 5.0 | 2.0 | 5.0 |
| Behenyl alcohol | 3.0 | 2.0 | 1.0 | 1.0 | — |
| Stearyl alcohol | — | 2.0 | 1.0 | 2.0 | 1.75 |
| Cetyl stearyl alcohol | 3.0 | — | 1.0 | — | 2.0 |
| Myristyl alcohol | 0.5 | 0.75 | 1.0 | 2.0 | 0.25 |
| Calendula oil | 5 | 1.0 | 1.5 | 2.0 | 0.75 |
| Trilaureth-4 phosphate | 0.5 | 1.0 | 0.75 | 3.0 | 0.25 |
| Glycerol | 5.0 | 25 | 35 | 26 | 55 |
| Panthenol | 0.5 | 1.0 | 0.75 | 0.25 | 0.1 |
| Perfume | q.s. | q.s. | q.s. | q.s. | q.s. |
| Methyl paraben | 0.4 | 0.1 | 0.05 | 0.3 | 0.4 |
| Propyl paraben | 0.3 | 0.4 | 0.25 | 0.15 | — |
| Sorbitol | 5.0 | — | 1.0 | — | 5.0 |
| Urea | 10.0 | — | — | 3.0 | — |
| Water | ad 100 | ad 100 | ad 100 | ad 100 | ad 100 |

### O/W Emulsion

| | 11 | 12 | 13 | 14 | 15 |
|---|---|---|---|---|---|
| Glyceryl stearate | 1.0 | 0.5 | 0.1 | 4.5 | 0.3 |
| Polyethylene- | 10.0 | 1.0 | 5 | — | — |
| glycol(20)cetearylether | | | | | |
| Triglycerin | — | — | — | — | 2.5 |
| methylglucose distearate | | | | | |
| Glyceryl laurate | 1.0 | 2.0 | 3.0 | 0.5 | 5.0 |
| Cyclomethicone | — | 2.0 | — | 1 | — |
| Dimethicone | 0.5 | 3.0 | 0.75 | 1.5 | 0.2 |
| Behenyl alcohol | 1.0 | 2.0 | 2 | 1 | 0.75 |
| Dicaprylyl carbonate | 3.0 | 5.0 | 1.0 | 15.0 | 5.0 |
| Stearyl alcohol | 2.0 | — | 3.0 | 1 | 0.75 |
| Cetylstearyl alcohol | 1.0 | 3.0 | 1.0 | 2.5 | 2.5 |
| Tocopherol | 0.5 | 0.5 | 0.75 | 0.25 | 0.1 |
| Octyldodecanol | 0.5 | — | 0.75 | 3.0 | 0.25 |
| Panthenol | 0.5 | 5.0 | 0.75 | 0.25 | 0.1 |
| Carbomer | 0.05 | 0.35 | 0.15 | 0.1 | — |
| Perfume | q.s. | q.s. | q.s. | q.s. | q.s. |
| Caprylic/Capric | 1.0 | 5.0 | 3.0 | 5.0 | 10.0 |
| triglyceride | | | | | |
| Methyl paraben | 0.4 | 0.3 | 0.05 | 0.3 | 0.4 |
| Propyl paraben | 0.3 | — | 0.25 | 0.15 | — |
| Calendula oil | 0.5 | 0.75 | 1.5 | 3.0 | 0.1 |
| Iodopropynylbutyl | — | — | 0.05 | — | 0.1 |
| carbamate | | | | | |
| Phenoxyethanol | — | 0.5 | — | 0.15 | — |
| Sorbitol | 10 | — | — | 5.0 | — |
| Butylene glycol | — | 10.0 | — | 5.0 | 10.0 |
| Propylene glycol | — | — | 10 | 5.0 | — |
| Glycerol | 5.0 | 7.5 | 15.0 | 5.0 | 35.0 |
| Water | ad 100 | ad 100 | ad 100 | ad 100 | ad 100 |

1.-23. (canceled)

24. An O/W emulsion, wherein the emulsion comprises (a) one or more fatty alcohols, (b) one or more glycerol esters of fatty acids, and at least 15% by weight, based on the total weight of the emulsion, of (c) at least one moisturizer.

25. The emulsion of claim 24, wherein (c) comprises glycerol.

26. The emulsion of claim 24, wherein the emulsion comprises at least 25% by weight of (c).

27. The emulsion of claim 24, wherein the (b) has a melting point of from about 32° C. to about 56° C.

28. The emulsion of claim 24, wherein the emulsion comprises at least about 0.1% by weight of (b).

29. The emulsion of claim 24, wherein the emulsion comprises not more than about 3% by weight of (b).

30. The emulsion of claim 24, wherein the emulsion comprises at least about 2% by weight of (a).

31. The emulsion of claim 24, wherein the emulsion comprises not more than about 10% by weight of (a).

32. The emulsion of claim 24, wherein (b) has a chain length of less than 22 carbon atoms.

33. The emulsion of claim 24, wherein (b) comprises glyceryl laurate.

34. The emulsion of claim 24, wherein the emulsion further comprises (d) at least one phosphate emulsifier.

35. The emulsion of claim 34, wherein (d) comprises at least one of trilaureth-4 phosphate and triceteareth-4 phosphate.

36. The emulsion of claim 34, wherein the emulsion comprises at least about 0.1% by weight of (d).

37. The emulsion of claim 34, wherein the emulsion comprises not more than about 5% by weight of (d).

38. The emulsion of claim 24, wherein the emulsion further comprises one or more substances selected from skin care, wound-healing, anti-inflammatory and pain-relieving substances.

39. The emulsion of claim 24, wherein the emulsion further comprises (e) candelula oil.

40. The emulsion of claim 39, wherein the emulsion comprises from about 0.01% to about 5% by weight of (e).

41. The emulsion of claim 24, wherein the emulsion further comprises (f) panthenol.

42. The emulsion of claim 41, wherein the emulsion comprises from 0.1% to 5% by weight of (f).

43. The emulsion of claim 24, wherein the emulsion comprises glycerol, panthenol and calendula oil.

44. An O/W emulsion, wherein the emulsion comprises based on the total weight of the emulsion, from about 2% to about 10% of (a) one or more fatty alcohols, from about 0.5% to 3% by weight of (b) one or more glycerol esters of fatty acids which comprise glyceryl laurate, and at least 20% by weight of (c) at least one moisturizer which comprises glycerol.

45. The emulsion of claim 44, wherein the emulsion further comprises from about 0.5% to 3% by weight of (d) one or more phosphate emulsifiers.

46. The emulsion of claim 45, wherein (d) comprises at least one of trilaureth-4 phosphate and triceteareth-4 phosphate.

47. The emulsion of claim 46, wherein the emulsion further comprises panthenol and calendula oil.

48. A cosmetic composition which comprises the emulsion of claim 24.

49. A method of improving and/or enhancing skin moisture and/or skin smoothness, wherein the method comprises applying to the skin the emulsion of claim 24.

50. A method of improving the consistency and/or stickiness of a cosmetic preparation, wherein the method comprises incorporating into the preparation one or more fatty alcohols, one or more glycerol esters of fatty acids, and at least 15% by weight of at least one moisturizer.
